(19) United States
(12) Patent Application Publication
(10) Pub. No.: US 2024/0225652 A1
(43) Pub. Date: Jul. 11, 2024

(54) RETRACTABLE TROCAR FOR CIRCULAR STAPLING INSTRUMENTS

(71) Applicant: Covidien LP, Mansfield, MA (US)

(72) Inventors: Yishuai Xu, Shanghai (CN); Li Xiang

(21) Appl. No.: 18/563,166

(22) PCT Filed: Jun. 9, 2021
(86) PCT No.: PCT/CN2021/099102

§ 371 Date: Nov. 21, 2023

(51) Int. Cl. A61B 17/115; A61B 17/11; A61B 17/34

(52) U.S. Cl. CPC A61B 17/1155; A61B 17/3417; A61B 2017/1132

(57) ABSTRACT

A trocar member for a circular stapling instrument includes a tubular housing and a tip member moveable relative to the tubular housing between an extended position and a retracted position. The tubular housing includes proximal and distal portions and defines a longitudinal passage. The tubular housing includes a flange configured for operable engagement with an anvil assembly. The tip member is disposed entirely within the tubular housing when in the retracted position.

### FIELD

The disclosure relates to circular stapling instruments. More particularly, this disclosure relates to circular stapling instruments including a trocar assembly having a retractable trocar for facilitating attachment of an anvil assembly to the circular stapling instrument.

### BACKGROUND

Circular stapling instruments are used to perform end to end anastomosis. During a typical end to end anastomosis procedure, an anvil assembly of the circular stapling instrument is secured to a first section of a tubular organ and a loading unit of the circular stapling instrument is secured to a second section of the tubular organ. The anvil assembly is then attached to a trocar member extending from the loading unit. The trocar member includes a piercing tip to facilitate positioning the second section of the tubular organ about the loading unit. Attaching the anvil assembly to the trocar member requires aligning a center rod assembly of the anvil assembly with the trocar member, which, in the narrow confines of the abdominal cavity, may require stretching of tissue and pose a potential risk to the tissue.

FIG. 1 illustrates tension “T” that is created when aligning a center rod “CR” of an anvil assembly “AA” with a trocar member “TM” extending from a loading unit “LU” of a circular stapling instrument “CS”. To attach the anvil assembly “AA” to the center rod “CR”, the anvil assembly “AA” is placed end to end with the trocar member “TM”, requiring a space within the abdominal cavity at least as long as the combined length of the trocar member “TM” and the anvil assembly “AA”.

To better facilitate attachment of an anvil assembly to a trocar member of the circular stapling instrument, it would be beneficial to have a trocar member having a length that may be shortened prior to the anvil assembly being attached to the trocar member.

### SUMMARY

A trocar member for a circular stapling instrument includes a tubular housing and a tip member moveable relative to the tubular housing between an extended position and a retracted position. The tubular housing includes proximal and distal portions and defines a longitudinal passage. The tubular housing includes a flange configured for operable engagement with an anvil assembly. The tip member is disposed entirely within the tubular housing when in the retracted position.

In certain aspects of the disclosure, the trocar member further includes a spring member for biasing the tip member to the retracted position. The trocar member may include a latch mechanism for maintaining the tip member in the extended position. The latch mechanism may include a latch member. The latch member may be movable between a locked position and an unlocked position. The latch member may be configured to engage the tip member when in the locked position to maintain the tip member in the extended position. The latch member may be spaced from the tip member when in the unlocked position to permit movement of the tip member from the extended position to the retracted position. The tip member may include a cylindrical body and an extension extending from the cylindrical body. The spring member may be a tension spring. The flange may be configured for operable engagement with a center rod assembly of the anvil assembly. The tip member may include a tissue piercing tip.

A trocar member for a circular stapling instrument includes a tubular housing having proximal and distal portions, and a tip member moveable within the longitudinal passage of the tubular housing between an extended position and a retracted position. The tubular housing defines a longitudinal passage. The tubular housing includes a flange configured for operable engagement with an anvil assembly. The trocar member includes a first length when the tip member is in the extended position and a second, shorter length when the tip member is in the retracted position.

In aspects of the disclosure, the trocar member further includes a spring member for biasing the tip member to the retracted position. The trocar member may include a latch mechanism for maintain the tip member in the extended position. The latch mechanism may include a latch member. The latch member may be movable between a locked position and an unlocked position. The latch member may be configured to engage the tip member when in the locked position to maintain the tip member in the extended position. The latch member may be spaced from the tip member when in the unlocked position to permit movement of the tip member from the extended position to the retracted position. The tip member may include a cylindrical body and an extension extending from the cylindrical body. The spring member may be a tension spring. The flange may be configured for operable engagement with a center rod assembly of the anvil assembly. The tip member may include a tissue piercing tip.

A circular stapling device includes an elongate body having proximal and distal portions, a loading unit supported on the distal portion of the elongate body, a trocar member extending through the loading unit. The trocar member includes a tubular housing and a tip member moveable relative to the tubular housing between an extended position and a retracted position. The tubular housing includes a flange configured for releasable connection with an anvil assembly.

### BRIEF DESCRIPTION OF THE DRAWINGS

The accompanying drawings, which are incorporated in and constitute a part of this specification, illustrate aspects of the disclosure and, together with a general description of the disclosure given above, and the detailed description given below, explain the principles of the disclosure, wherein:

FIG. 1 is a schematic side view of an anvil assembly of a traditional circular stapling instrument being attached to a trocar member of the circular stapling instrument;

FIG. 2 is a side view of a circular stapling instrument according to aspects of the disclosure;

FIG. 3 is an enlarged view of the indicated area of detail shown in FIG. 2;

FIG. 4 is a perspective side view of a trocar member of the circular stapling instrument shown in FIG. 2;

FIG. 5 is a perspective side view of the trocar member shown in FIG. 4, with parts separated;

FIG. 6 is an enlarged view of the indicated area of detail shown in FIG. 5;

FIG. 7 is a cross-sectional end view taken along section line 7-7 shown in FIG. 4;

FIG. 8 is a cross-sectional side view taken along section line 8-8 shown in FIG. 3;

FIG. 9 is an enlarged view of the indicated area of detail shown in FIG. 8;

FIG. 10 is the cross-sectional side view shown in FIG. 8 with a tip member of the trocar member in a retracted position;

FIG. 11 is an enlarged view of the indicated area of detail shown in FIG. 10;

FIG. 12 is an enlarged view of the indicated area of detail shown in FIG. 11;

FIG. 13 is a perspective side view of a distal end of the circular stapling instrument shown in FIG. 2, with a tip member of the trocar member in a retracted position and an anvil assembly spaced from the trocar member;

FIG. 14 is a perspective top view of the trocar member shown in FIG. 13, with the tip member in a retracted position;

FIG. 15 is the perspective side view of the distal end of the circular stapling instrument shown in FIG. 13, with the anvil assembly attached to the trocar member; and

FIG. 16 is a cross-sectional side view taken along section line 16-16 shown in FIG. 15.

### DETAILED DESCRIPTION

Aspects of the disclosure are described in detail with reference to the drawings, in which like reference numerals designate identical or corresponding elements in each of the several views. As used herein, the term “distal” refers to that portion of the adapter assembly or surgical device, or component thereof, farther from the user during use of the instrument in its customary fashion, while the term “proximal” refers to that portion of the adapter assembly or surgical device, or component thereof, closer to the user during use of the instrument in its customary fashion. As used herein, the term clinician refers to anyone involved in a surgical procedure, including but not limited to, surgeons, support staff, and other medical personnel. As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

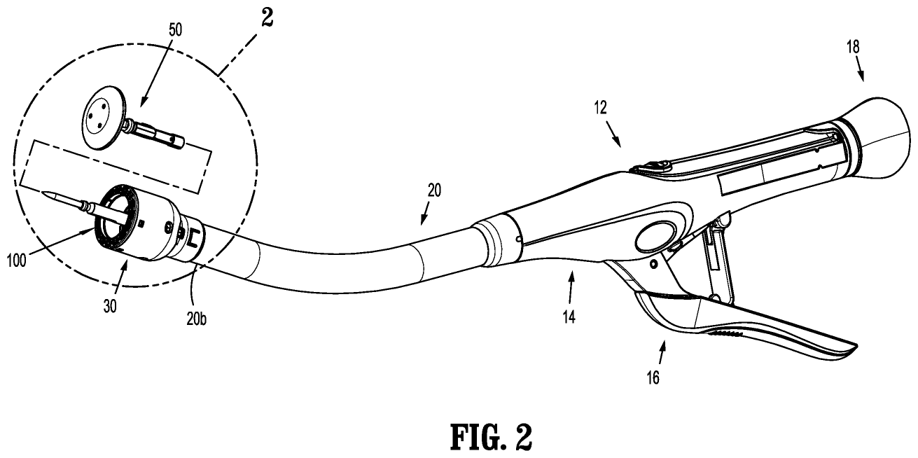

FIG. 2 illustrates a circular stapling instrument according to aspects of the disclosure, shown generally as circular stapler 10. The circular stapler 10 includes a manually actuated handle assembly 12 including a housing 14, a trigger assembly 16, and an approximation knob 18. An elongate body 20 extends from the handle assembly 12 and includes a loading unit 30 disposed on a distal end 20b of the elongate body 20. Although shown as being integrally formed with the handle assembly 12, it is envisioned that the elongate body 20 may form a separate and/or removable component. Similarly, the loading unit 30 may be integrally formed with the elongate body 20 or configured as a replaceable unit.

A trocar assembly 100 extends from the elongate body 20 of the circular stapler 10 through the loading unit 30. The trocar assembly 100 is movable between advanced and retracted positions. In the advanced position, the trocar assembly 100 is configured to facilitate securing a section of tubular organ about the loading unit 30 and facilitate attaching the anvil assembly 50 to the elongate body 20. When an anvil assembly 50 is coupled to the trocar assembly 100, movement of the trocar assembly 100 to the retracted position causes the anvil assembly 50 to move towards the loading unit 30, thereby compressing tissue between the anvil assembly 50 and the loading unit 30 prior to stapling and cutting of the tissue. As will be described in further detail below, the trocar assembly 100 is adapted to be releasably coupled to the anvil assembly 50.

The circular stapler 10 will only be described to the extent necessary to fully disclose aspects of the disclosure. For a detailed description of exemplary circular staplers with manually actuated handle assemblies, please refer to U.S. Pat. Nos. 7,364,060, and 7,303,106, the contents of which are incorporated herein by reference herein their entirety.

Although aspects of the trocar assembly 100 will be described in detail with respect to the manually actuated handle assembly 12, it is envisioned that aspects of the disclosure may be modified for use with powered and non-powered handle assemblies having similar and various other configurations, and for use with robotic systems. For a detailed description of exemplary electromechanical powered handle assemblies, please refer to U.S. Pat. Nos. 9,055,943 and 9,918,713, the contents of which are incorporated by reference herein in their entirety.

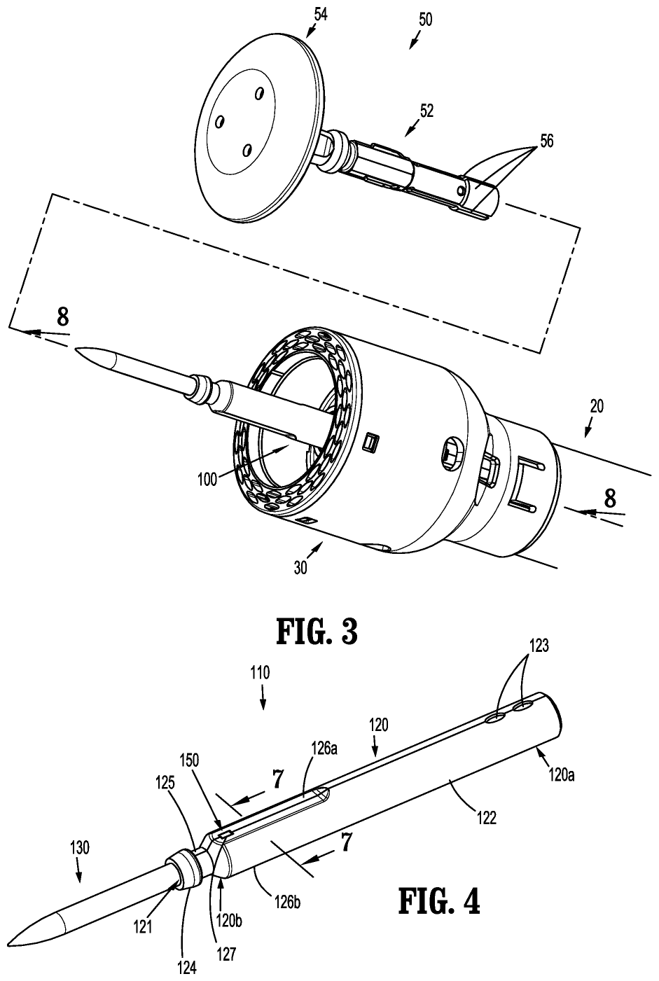

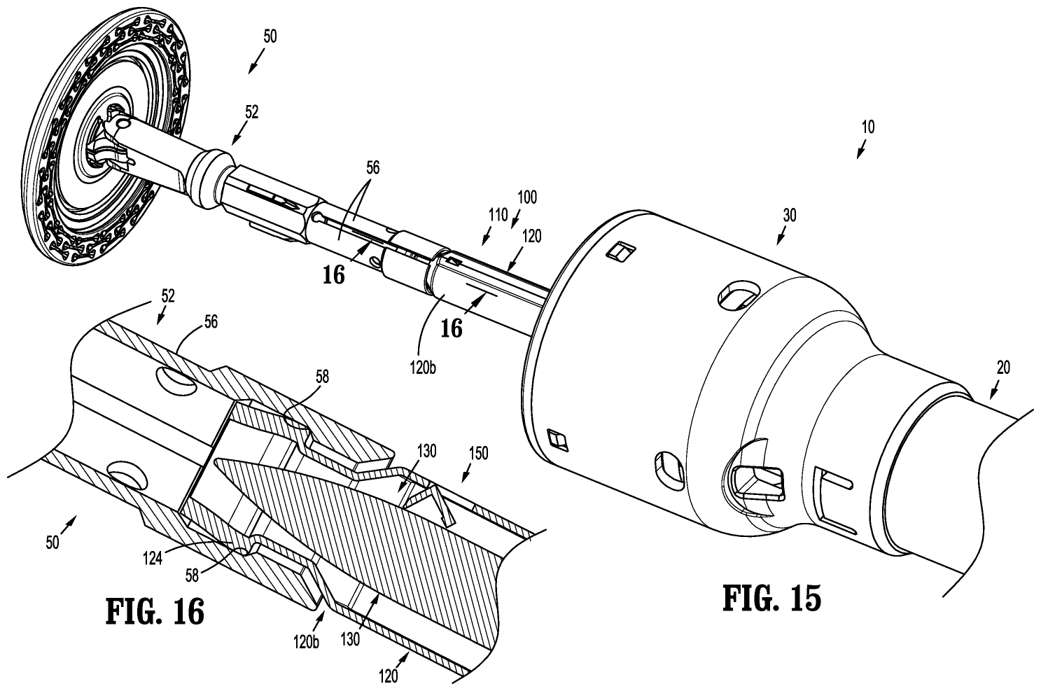

FIG. 3 illustrates the distal or operational end of the circular stapler 10 including the loading unit 30, a trocar member 110 of the trocar assembly 100 extending through and from the loading unit 30, and the anvil assembly 50 detached from the trocar assembly 100. The anvil assembly 50 includes a center rod assembly 52 and a head assembly 54 pivotally secured to the center rod assembly 52. A free end of the center rod assembly 52 includes a plurality of legs 56 each having a shoulder 58 (FIG. 15). The anvil assembly 50 will only be described to the extent necessary to fully disclose the aspects of the disclosure. For a detailed description of an exemplary anvil assembly, please refer to U.S. Pat. No. 8,328,063, the content of which is incorporated by reference herein in its entirety.

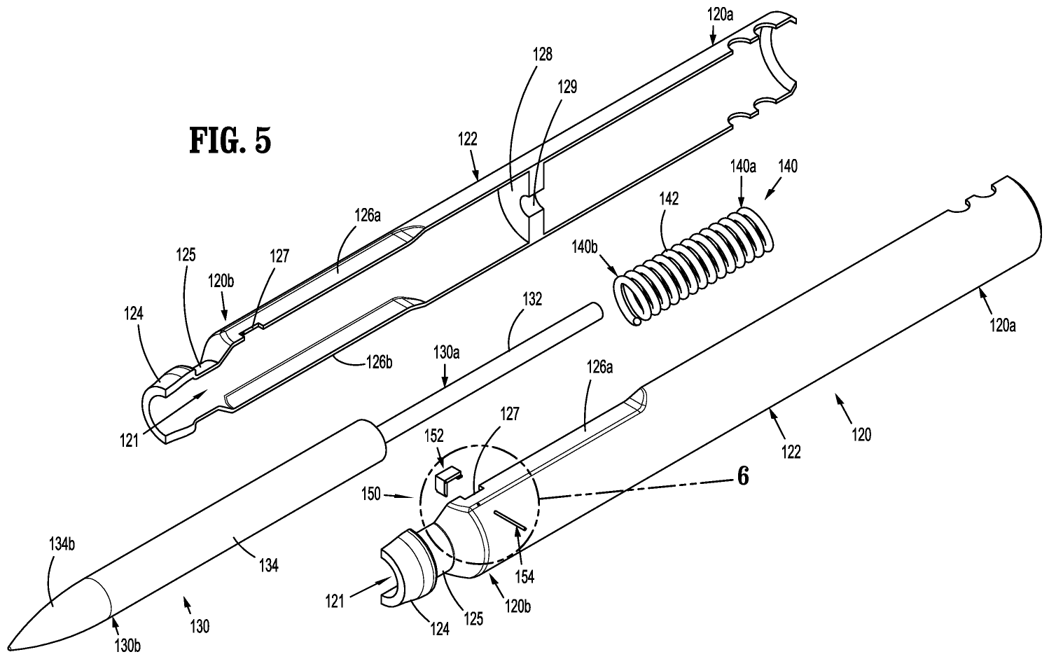

FIGS. 4 and 5 illustrate the trocar member 110 of the trocar assembly 100 including a housing 120 and a tip member 130 extendable from the housing 120. As will be described in further detail below, the tip member 130 is retained against the bias of a spring member 140 (FIG. 5) by a latch mechanism 150.

The housing 120 of the trocar member 110 defines a substantially tubular body 122 having proximal and distal portions 120a, 120b and defining a longitudinal passage 121. The proximal portion 120a of the housing 120 is configured to engage a retractable connector member 60 (FIG. 9) that is operably connected with the approximation knob 18 (FIG. 2) of the handle assembly 12 of the circular stapler 10 to permit retraction of the trocar member 110 relative to the loading unit 30. As shown, the proximal end 120a of the tubular body 122 includes a pair of openings 123 each configured to receive a locking pin 62 (FIG. 9) or other locking mechanism for securing the trocar member 110 to the connector member 60.

A flange 124 is formed on the distal portion 120b of the housing 120 of the trocar member 110 and defines a groove 125. The flange 124 and groove 125 are configured to facilitate connection with the anvil assembly 50. More particularly, the shoulder 58 on each of the plurality of legs 56 of the center rod assembly 52 of the anvil assembly 50 engage the flange 124 of the trocar member 110 to secure the anvil assembly 50 relative to the trocar member 110. As is known in the art, retraction of the trocar assembly 100 within the loading unit 30 secures the plurality of legs 56 of the center rod assembly 52 of the anvil assembly 50 relative to the trocar member 110, thereby locking the anvil assembly 50 to the trocar member 110. The distal portion 120b of the housing 120 further includes opposed first and second flattened surfaces 126a, 126b. A cutout 127 is located along the first flattened surface 126a. A shelf 128 (FIG. 5) is positioned between the proximal and distal portions 120a, 120b of the housing 120 of the trocar member 110 and defines an opening 129.

The tip member 130 of the trocar member 110 includes a proximal portion 130a (FIG. 5) and a distal portion 130b. The proximal portion 130a of the tip member 130 includes a cylindrical extension 132 sized to be received through the opening 129 in the shelf 128 of the housing 120. The distal portion 130b of the tip member 130 includes a cylindrical body 134 having a tapered, tissue piercing free end 134b. As will be described in detail below, the tip member 130 is configured to be retracted within the housing 120 of the trocar assembly 100.

The trocar assembly 100 includes a spring member 140 in the form of a tension spring 142 for retracting the tip member 130 of the trocar member 110 within the housing 120 of the trocar member 110. Although shown as a tension spring 142, it is envisioned that the spring member 140 may include elastic bands, pistons, or other mechanisms suitable for retracting the tip member 130 within the housing 120. The spring member 140 is configured to be received about the cylindrical extension 132 of the tip member 130 and includes proximal and distal end portions 140a, 140b. The proximal end portion 140a of the spring member 140 is configured to be secured relative to the shelf 128 in the housing 120 and the distal end portion 140b of the spring member 140 is configured to be secured relative to the cylindrical body 134 of the tip member 130. In this manner, movement of the tip member 130 away from the shelf 128 causes the spring member 140 to stretch, thereby creating a biasing force against the spring member 140.

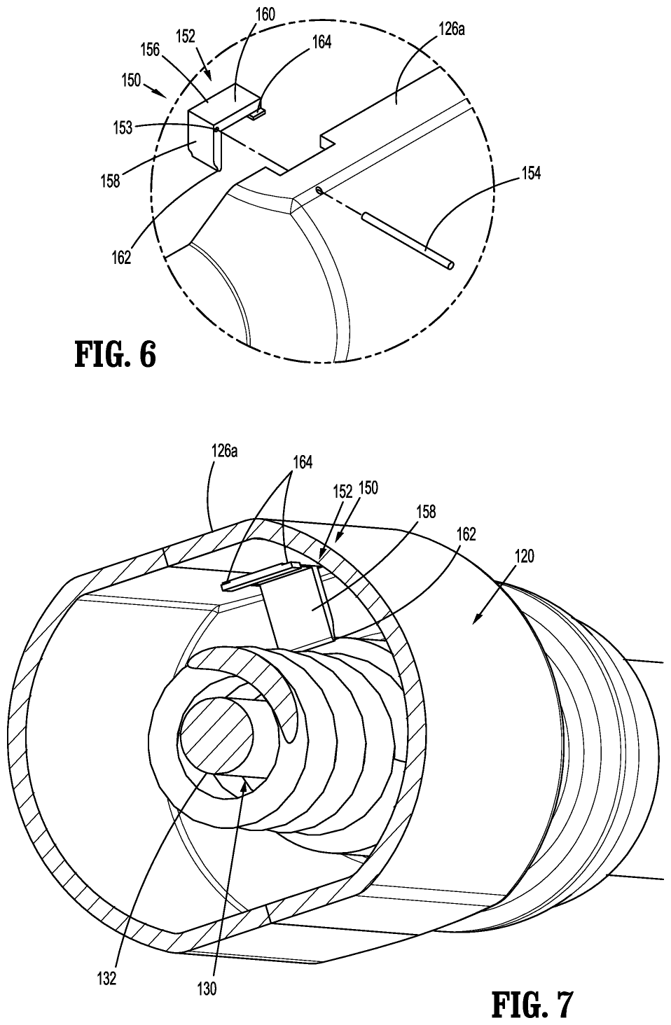

FIG. 6 illustrates the latch mechanism 150 of the trocar assembly 100 including a latch member 152 and a pin 154 for pivotally securing the latch member 152 within the cutout 127 (FIG. 5) in the first flattened surface 126a of the housing 120 of the trocar member 110. The latch member 152 includes a substantially L-shaped body 156 having a first leg 158 and a second leg 160. An engaging portion 162 is formed on the first leg 158 and a pair of tabs 164 extend outwardly from second leg 160. The engaging portion 162 on the first leg 158 is configured to engage the cylindrical body 134 of the tip member 130 when the latch mechanism 150 is in a locked position (FIG. 9). The pair of tabs 164 on the second leg 160 engage an underside of the first flattened surface 126a of the housing 120 of the trocar member 110 to retain the latch member 152 within the housing 120 of the trocar member 110. The latch member 152 defines an opening 153 between the first and second legs 158, 160 of the substantially L-shaped body 154 for receiving the pin 154.

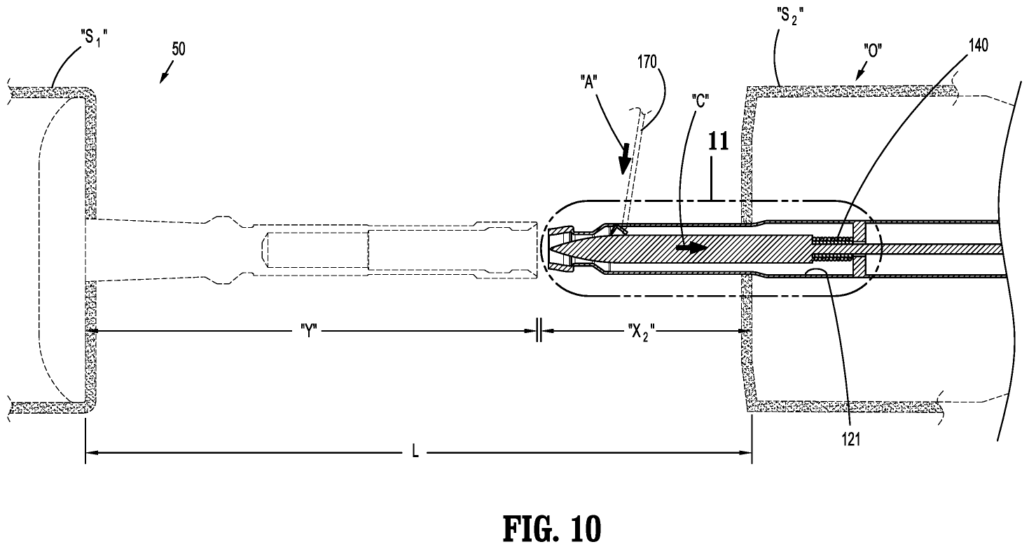

FIGS. 7-9 illustrate the trocar member 110 of the trocar assembly 100 with the tip member 130 in the extended position and the latch mechanism 150 in the locked position. When the tip member 130 of the trocar member 110 is in the extended position, the tension spring 140, with the proximal end portion 140a secured relative to the shelf 126 in the housing 120 and the distal end portion 140b secured relative to the cylindrical body 134 of the tip member 130, is stretched to create a biasing force against the tip member 130. Engagement of the engaging portion 162 of the latch member 152 of the latch mechanism 150 with the cylindrical body 134 of the tip member 130 of the trocar member 110 locks the tip member 130 in the extended position. When the tip member 130 of the trocar member 110 is in the extended position, the trocar member 110 operates as a traditional trocar member, facilitating the positioning of a second section “S2” (FIG. 10) of a tubular organ “O” receiving the anastomosis about the loading unit 30.

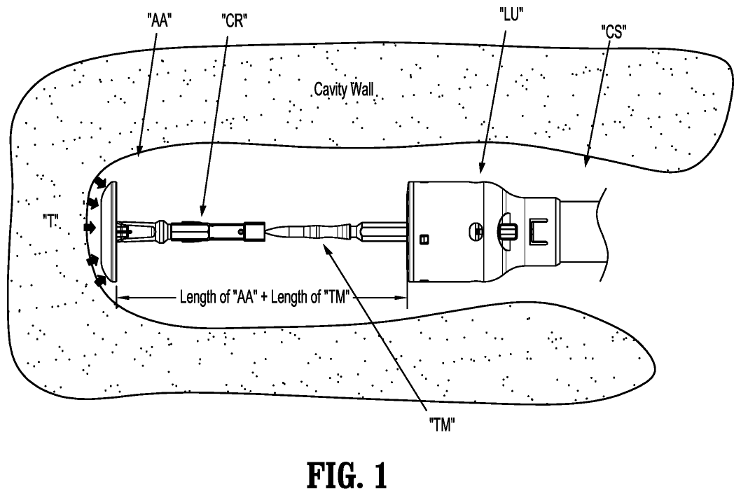

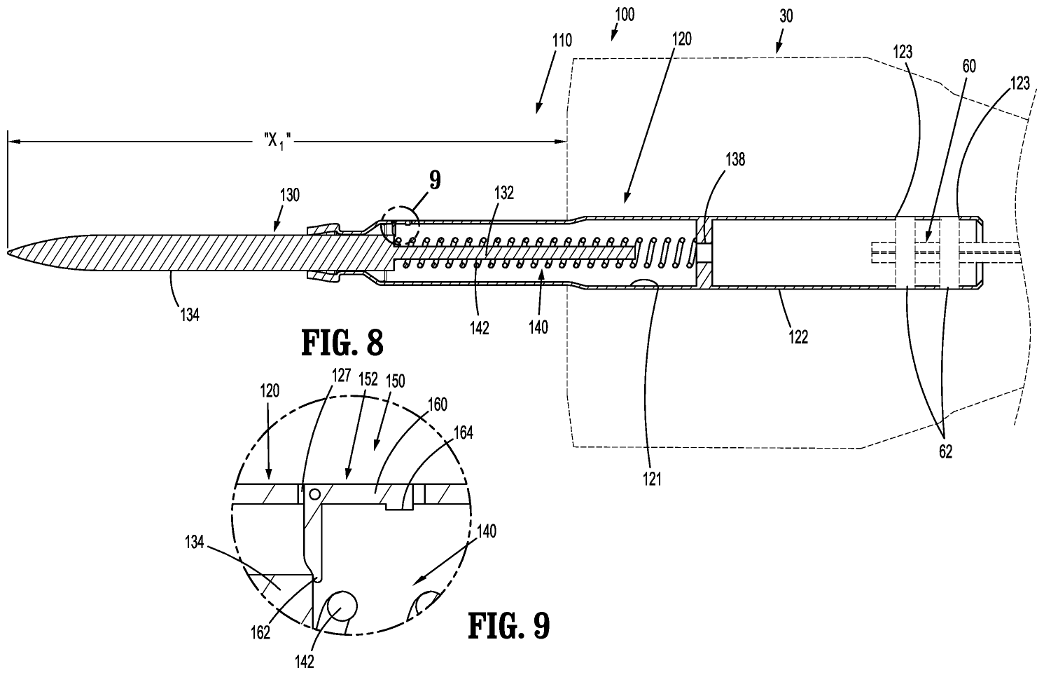

After the second section “S2” of the tubular organ “O” receiving the anastomosis is positioned about the loading unit 30 and prior to the tip member 130 of the trocar member 110 moving to the retracted position, the distance between loading unit 30 and a distalmost end of the trocar member 110 defines a first distance “x1” (FIG. 8). As described previously, attaching the anvil assembly 50 (FIG. 3) to a traditional trocar member “TM” (FIG. 1) requires a space equal to at least the first distance “x1” plus the length of the anvil assembly 50, a distance “y” (FIG. 10).

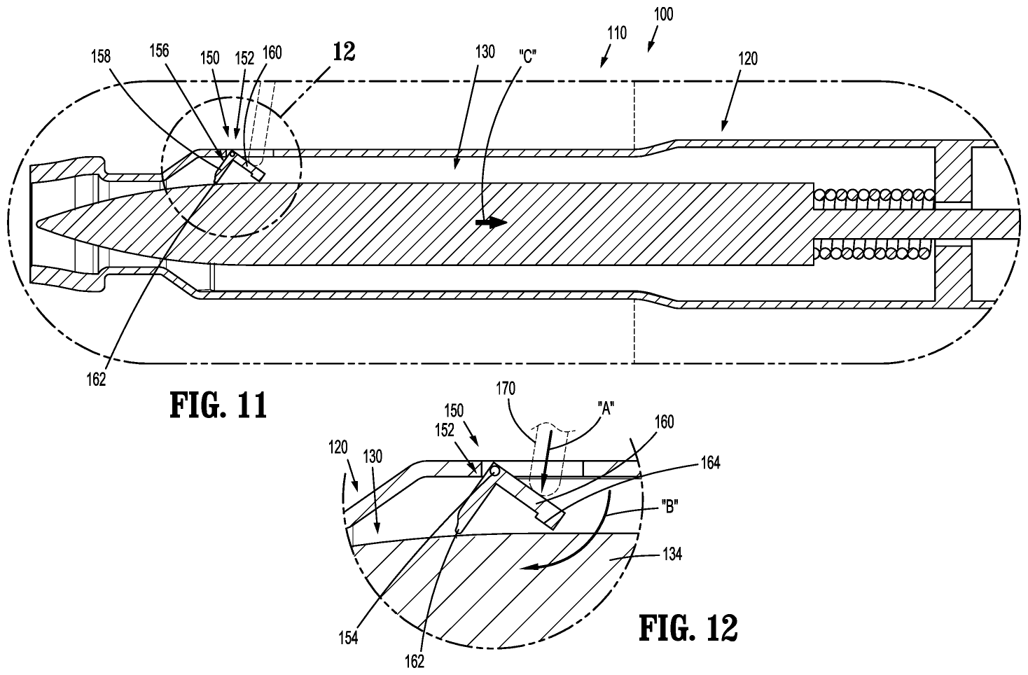

FIGS. 10-12 illustrate the trocar member 110 of the trocar assembly 100 subsequent to the second section “S2” of the tubular organ being positioned about the loading unit 30 and prior to attaching the anvil assembly 50 to the trocar member 110. As noted previously, the tissue piercing free end 134b of the distal portion 130b of the tip member 130 is configured to facilitate positioning of the second section “S2” of the tubular organ about the loading unit 30. Once the second section “S2” is positioned about the loading unit 30, the tip member 130 of the trocar member 110 is no longer necessary and may be retracted. Retraction of the tip member 130 requires pressing on the second leg 160 of the latch member 152 of the latch mechanism 150, as indicated by arrow “A” in FIG. 10, to cause the latch member 152 to pivot about pivot pin 154, as indicated by arrow “B” in FIG. 12, to move the latch member 152 from a locked position to an unlocked position. As the latch member 152 of the latch mechanism 150 pivots to the unlocked position, the engaging portion 160 of the latch member 152 disengages from the cylindrical body 134 of the tip member 130, allowing the tension spring 142 to retract the tip member 130 within the housing 120 of the trocar assembly 100, as indicated by arrow “C” in FIG. 11.

Although the latch member 152 of the latch mechanism 150 is shown as being pressed directly by a device 170, it is envisioned that that the latch mechanism 150 may have a release button or actuation mechanism that may be depressed directly by the clinician. It is further envisioned that the anvil assembly 50 may be modified to include a mechanism for effecting unlocking of the latch mechanism 150. It also envisioned that a release mechanism may be disposed in the handle assembly 12 (FIG. 2) to permit remote release of the latch mechanism 150. In this manner, a wire or other suitable means may extend through the elongate body 20 (FIG. 2) of the circular stapler 10 and include a release button in the handle assembly 12 that may be triggered by the clinician to cause the unlocking of the latch mechanism 150 and the subsequent retraction of the tip member 130.

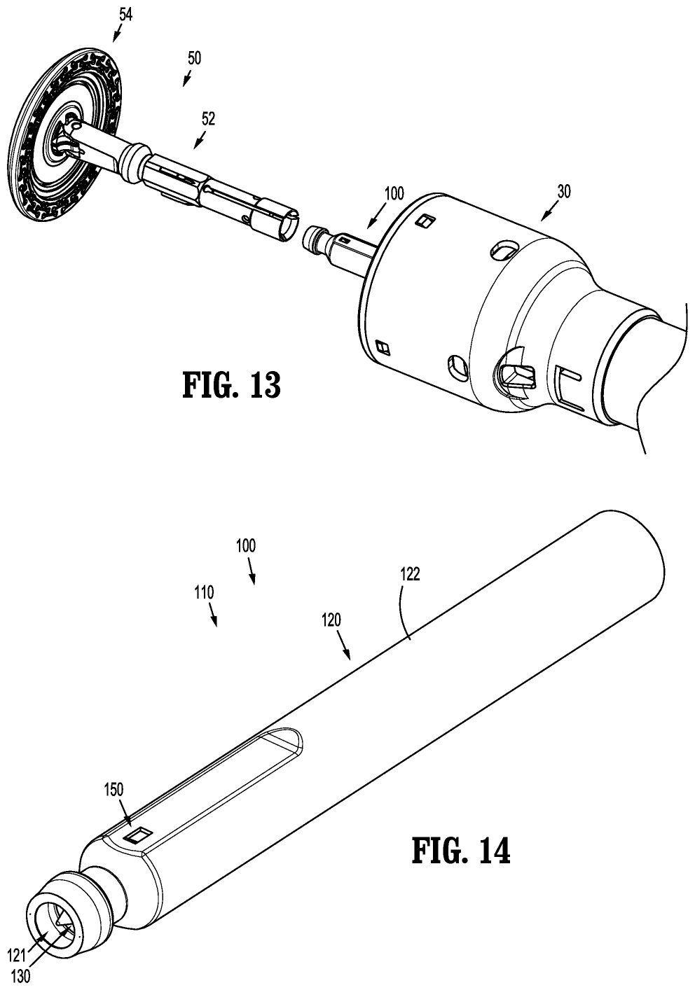

FIGS. 13 and 14 illustrate the trocar member 110 with the tip member 130 in the retracted position, disposed entirely within the longitudinal passage 121 of the housing 120. The tip member 130 is maintained in the retracted position by the bias of the tension spring 140 (FIG. 10). It is envisioned that the latch mechanism 150 may be configured such that the latch member 152 retains the tip member 130 of the trocar member 110 in the retracted position.

After the tip member 130 of the trocar member 130 is retracted within the housing 120 of the trocar member 110, a distalmost end of the trocar member 110 defines a second distance “x2” (FIG. 10). The second distance “x2” is shorter than the first distance “x1”. In this manner, the space required to align the center rod assembly 52 of the anvil assembly 50 with the trocar member 110 when the trocar member 110 is in the retracted position (FIG. 10) is less than the space required to align the center rod assembly 52 of the anvil assembly 50 with the trocar member 110 when the trocar member 110 is in the extended position (FIG. 8). In certain aspects of the disclosure, the combined length of the anvil assembly 50 and the first distance “x1” is ˜91 mm and the combined length of the anvil assembly 50 and the second distance “x2” is ˜67 mm, resulting in a decrease in combined length of about 26.4%. As noted previously, the shorter length required to align the center rod assembly 52 of the anvil assembly 50 with the trocar member 110 of the circular stapler 10 will reduce the space necessary to complete the procedure and reduce the likelihood of damage to the tissue being stapled and the surrounding tissue.

FIGS. 15 and 16 illustrate the anvil assembly 50 attached to the trocar member 110 of the trocar assembly 100. In this configuration, the shoulder 58 of each the plurality of legs 56 of the center rod assembly 52 of the anvil assembly 50 engage the flange 124 of on the distal portion 120b of the housing 120 of the trocar member 110 to secure the anvil assembly 50 relative to the trocar member 110. As noted previously, retraction of the trocar assembly 100 into the elongate body 30 of the circular stapler 10 locks the center rod assembly 52 of the anvil assembly 50 to the trocar member 110.

Once the anvil assembly 50 is secured to the trocar assembly 100, the circular stapler 10 operates in as is known in the art and the anastomosis procedure is completed in a traditional manner. It is envisioned that the trocar member 110 may be configured for sterilization and reuse.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. A trocar member for a circular stapling instrument, the trocar member comprising:
a tubular housing having proximal and distal portions and defining a longitudinal passage, the tubular housing including a flange configured for operable engagement with an anvil assembly; and
a tip member moveable relative to the tubular housing between an extended position and a retracted position, wherein the tip member is disposed entirely within the tubular housing when in the retracted position.

2. The trocar member of claim 1, including a spring member for biasing the tip member to the retracted position.

3. The trocar member of claim 2, including a latch mechanism for maintaining the tip member in the extended position.

4. The trocar member of claim 3, wherein the latch mechanism includes a latch member.

5. The trocar member of claim 4, wherein the latch member is movable between a locked position and an unlocked position.

6. The trocar member of claim 5, wherein the latch member is configured to engage the tip member when in the locked position to maintain the tip member in the extended position.

7. The trocar member of claim 6, wherein the latch member is spaced from the tip member when in the unlocked position to permit movement of the tip member from the extended position to the retracted position.

8. The trocar member of claim 1, wherein the tip member includes a cylindrical body and an extension extending from the cylindrical body.

9. The trocar member of claim 2, wherein the spring member is a tension spring.

10. The trocar member of claim 1, wherein the flange is configured for operable engagement with a center rod assembly of the anvil assembly.

11. The trocar member of claim 1, wherein the tip member includes a tissue piercing tip.

12. A trocar member for a circular stapling instrument, the trocar member comprising:
a tubular housing having proximal and distal portions and defining a longitudinal passage, the tubular housing including a flange configured for operable engagement with an anvil assembly; and
a tip member moveable within the longitudinal passage of the tubular housing between an extended position and a retracted position, wherein the trocar member includes a first length when the tip member is in the extended position and a second, shorter length when the tip member is in the retracted position.

13. The trocar member of claim 12, including a spring member for biasing the tip member to the retracted position.

14. The trocar member of claim 13, including a latch mechanism for maintain the tip member in the extended position.

15. The trocar member of claim 14, wherein the latch mechanism includes a latch member.

16. The trocar member of claim 15, wherein the latch member is movable between a locked position and an unlocked position.

17. The trocar member of claim 16, wherein the latch member is configured to engage the tip member when in the locked position to maintain the tip member in the extended position.

18. The trocar member of claim 17, wherein the latch member is spaced from the tip member when in the unlocked position to permit movement of the tip member from the extended position to the retracted position.

19. The trocar member of claim 12, wherein the distal portion of the tubular housing includes a flange configured for operable engagement with a center rod assembly of an anvil assembly.

20. A surgical stapling device comprising:
an elongate body having proximal and distal portions;
a loading unit supported on the distal portion of the elongate body; and
a trocar member extending through the loading unit, the trocar member including a tubular housing and a tip member moveable relative to the tubular housing between an extended position and a retracted position, the tubular housing including a flange configured for releasable connection with an anvil assembly.
